(19) United States
(12) Patent Application Publication
(10) Pub. No.: US 2010/0166814 A1
(43) Pub. Date: Jul. 1, 2010

(54) Cosmetic Composition Containing At Least Two Osmolytes With A Moisturizing Or Antiaging Effect

(75) Inventors: Marc C.M. Dumas, Saint Jean le Blanc (FR); Valérie A.S. Krzych, Les Bordes (FR); Delphine Pelle de Queral, Ingre (FR); Catherine Heusele, Limours (FR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LVMH Recherche, Saint Jean De Braye (FR)

(21) Appl. No.: 12/644,067

(22) Filed: Dec. 22, 2009

(30) Foreign Application Priority Data
Dec. 24, 2008 (FR) 0859083

(51) Int. Cl. A61K 8/60; A61K 8/14; A61Q 19/08; A61Q 3/02; A61Q 5/02; A61Q 5/12; A61K 8/97; A61K 8/96; A61K 8/64; A61K 8/99

(52) U.S. Cl. 424/401; 514/53; 424/61; 424/70.1; 424/722; 424/776; 424/767; 424/764; 424/758; 424/750; 424/769; 424/195.17; 514/17; 424/780

(57) ABSTRACT

The present invention relates to a cosmetic composition, which comprises a combination of at least two osmolytes chosen from the group comprising taurine or a derivative thereof, inositol, betaine and trehalose.

This composition is intended for restoring, maintaining or reinforcing the moisturization of the skin and/or for protecting it against different types of stress and/or for preventing or retarding the appearance of the signs of aging of the skin, or for attenuating the effects thereof, or alternatively for promoting cell or tissue longevity.

### CROSS REFERENCE TO RELATED APPLICATIONS

This application claims the benefit of French Patent Application No. 0859083, filed Dec. 24, 2008, the entirety of which is incorporated herein.

### TECHNICAL FIELD

The present invention relates to novel cosmetic compositions containing a combination of at least two osmolytes, and to its use as an active cosmetic agent, referred to hereinbelow as an “active agent”, in cosmetic compositions, especially moisturizing and/or antiaging compositions.

### BACKGROUND

Kosmotropic molecules are organic compounds that are specifically accumulated by the cell in response to a hyperosmotic stress such as dehydration, and which are then rapidly released therefrom (Kwon and Handler, Current Opin. Cell. Biol.; 1995; 7: 465-471 and Haussinger 1996; Biochem. J. 313: 697-710).

These compounds, which are also known under the generic term “osmolytes”, perturb the cell very little even at high concentrations and, furthermore, do not interfere with the functions of the proteins of said cells (Burg et al., Annu. Rev. Physiol. 1997; 59: 437-455).

WO 91/914 435 discloses a method for treating osmotic disorders, comprising the administration of an effective amount of osmolytes or precursors.

WO 03/005 979 discloses the use, in a cosmetic composition, of at least one osmolyte for treating or preventing impairments of cutaneous homeostasis, especially for caring for, treating or preventing dry skin or sensitive skin.

None of these publications discloses the synergistic combination of osmolytes that is the subject of the present invention, or reveals the noteworthy cosmetic properties demonstrated for said combination, especially in the field of moisturization and/or protection and/or antiaging.

### SUMMARY

The present invention thus relates to a cosmetic composition which comprises at least one cosmetic active agent comprising a combination of at least two osmolytes, and also to a novel use of a combination of at least two osmolytes as active agent in a cosmetic composition.

Said combination makes it possible especially to restore, maintain or reinforce the moisturization of the skin and/or to protect it against different types of stress and/or to prevent or retard the appearance of the signs of aging of the skin, or to attenuate the effects thereof, or alternatively to promote cell or tissue longevity.

The present invention also relates to a cosmetic care method for moisturizing the skin or for producing an anti-stress or antiaging protective effect, comprising such a combination of at least two osmolytes as active agent in a cosmetic composition.

### DETAILED DESCRIPTION OF ILLUSTRATIVE EMBODIMENTS

The main aim of the present invention is to provide a novel cosmetic active agent, referred to hereinbelow as an “active agent”, in a cosmetic composition especially having a moisturizing and/or protecting and/or antiaging effect.

A main aim of the present invention is also to provide a novel active agent of a cosmetic composition that has a good capacity for restoring, maintaining or reinforcing the moisturization of the skin and/or for protecting it against different types of stress and/or for preventing or retarding the appearance of the signs of aging of the skin, or for attenuating the effects thereof, or alternatively for promoting cell or tissue longevity.

A main aim of the present invention is also to provide a cosmetic care method for moisturizing the skin or for producing an anti-stress protective effect or an antiaging effect by means of the abovementioned novel active agent.

A main aim of the present invention is also to provide a novel cosmetic active agent that is compatible with repeated application to the skin without any significant side effects, especially any skin irritation.

An aim of the invention is also to solve the technical problem via a solution that is particularly simple, relatively inexpensive and usable at the industrial scale.

A first subject of the present invention is a cosmetic composition which comprises at least one cosmetic active agent comprising a combination of at least two osmolytes chosen from the group comprising taurine or a derivative thereof, inositol, betaine and trehalose.

Among the taurine derivatives, mention is made of hypotaurine.

According to the invention, inositol is also referred to as myoinositol.

Betaine is, itself, known without discrimination as trimethylglycine.

The trehalose is preferably in the form of D-trehalose.

Among the combinations comprising at least two osmolytes, a combination comprising taurine, or a derivative thereof, and inositol is preferred.

According to one preferred embodiment, said combination comprises three osmolytes chosen from the group comprising taurine or a derivative thereof, inositol, betaine and trehalose.

Among these combinations, a preferred combination is formed from taurine or a derivative thereof, inositol and betaine.

According to another preferred embodiment, said combination comprises four osmolytes chosen from the group comprising taurine or a derivative thereof, inositol, betaine and trehalose.

Among these combinations, a preferred combination is formed from taurine, inositol, D-trehalose and betaine.

These combinations make it possible to stimulate, in an unexpected manner for a person skilled in the art, the synergistic expression of the chaperone protein HSPA1A in epidermal cells.

According to a first variant, the weight ratio of each osmolyte in the abovementioned combination relative to the weight of the osmolyte of the combination that is present in smallest amount is between 1 and 10, preferably between 1 and 5 and even more preferably between 1 and 2.

According to yet another embodiment variant of the invention, the weight ratio of each osmolyte of the combination comprising taurine or a derivative thereof, inositol, betaine and trehalose is between 1/1/1/1 and 1/2/2/2. The combination of osmolytes according to the invention proved to be synergistic and to afford unexpected effects, and may consequently be used as an active agent in a cosmetic composition also comprising at least one cosmetically acceptable excipient.

The cosmetic composition thus comprises an effective amount of said combination of osmolytes to obtain the desired cosmetic effect.

The composition thus preferentially comprises from 0.001% to 10% by weight and preferably from 0.01% to 2% by weight of the combination according to the invention.

According to yet another embodiment of the invention, the abovementioned composition is characterized in that the abovementioned combination of osmolytes is encapsulated in lipid vesicles such as unilamellar or multilamellar liposomes.

According to one preferred embodiment, the lipid vesicles are based on amphiphilic lipids containing phosphatidylcholine, and especially based on lecithins.

According to another preferred variant of the invention, the abovementioned composition is which comprises D-xylose in addition to the abovementioned combination.

The cosmetic compositions according to the invention may comprise one or more other cosmetic active agents.

The cosmetic properties demonstrated for the combination of osmolytes according to the invention may be improved in the presence of cosmetic active agents that have similar and/or complementary cosmetic effects.

Besides a combination of osmolytes as defined previously, the cosmetic composition according to the invention may thus comprise one or more other plant extracts or molecules with moisturizing properties, such as glycols, in particular glycerol, or natural polyols, natural or synthetic ceramides, hyaluronic acid or fragments thereof of smaller molecular weight, or alternatively all or some of the molecular constituents of natural moisturizing factor (NMF), for instance lactate, citrate, urea, a PCA (pyrrolidonecarboxylic acid) salt, Na⁺, K⁺, Ca²⁺ or Mg⁺ ions, serine, citrulline, alanine and threonine, or alternatively lipid constituents such as cholesterols and cholesteryl sulfate, and fatty acids, including omega-3, omega-6 and omega-9.

Among the other cosmetic agents that may also advantageously be used in the composition according to the invention, mention may be made more particularly of:
molecules or extracts that stimulate the biosynthesis of the cutaneous aquaporins, for instance an extract of Ajuga turkestanica, an extract of Vanda coerulea, retinoic acid and retinol, or alternatively a combination of an extract of Malva sylvestris and of an extract of Centella asiatica that is rich in heterosides of the type such as madecassoside and asiaticoside,
molecules or extracts that activate the biosynthesis of profilaggrin and of filaggrin, for instance an extract of Voandzeia subterranea seeds,
molecules or extracts that activate the biosynthesis of involucrin and transglutaminase of type 1, for instance polyphenols, in particular epigallocatechin-3 gallate and extracts containing it,
molecules or extracts that activate caspase 14. It will be noted that these molecules or extracts are involved in the degradation of filaggrin to form NMF components. Examples that will be mentioned include green tea polyphenols, in particular epigallocatechin-3 gallate and extracts containing it,
molecules or extracts that stimulate epidermal kallikreins. It will be noted that these molecules or extracts regulate desquamation at the surface of the skin. A particular example that will be mentioned is an extract of nopal,
molecules or extracts that stimulate the synthesis of epidermal lipids. It will be noted that these molecules or extracts play an essential role in the water-barrier effect of the stratum corneum and the suppleness of the skin. Examples that will be mentioned in particular include an extract of Helianthus annuus L. or Luffa cylindrica seeds, and/or antioxidants that protect these lipids, for instance tocopherol and derivatives thereof, in particular esters such as the palmitate and the acetate,
molecules or extracts that reinforce the skin barrier by stimulating the terminal differentiation of keratinocytes and/or transglutaminase, in particular beta-ecdysone or calcium derivatives such as calcium gluconate,
molecules or extracts that stimulate epidermal renewal, in particular corn oil, and/or that protect the germinative compartment and the stem cells of the epidermis, for instance tocopheryl phosphate,
molecules or extracts that stimulate the expression of the beta-1 integrins and/or that promote the adhesion of the germinative keratinocytes to the dermo-epidermal junction, for instance magnesium aspartate or manganese chloride,
molecules or extracts that promote the formation of tight junctions. It will be noted that these molecules or extracts thus limit intercellular water losses. A particular example that will be mentioned is an extract of Castanea sativa,
molecules or extracts that stimulate the expression of the receptor cd44 and the binding of its natural ligand, hyaluronic acid, for instance calcium gluconate,
molecules or extracts that stimulate the synthesis of glycosaminoglycans (GAG) in the epidermis, especially a xylose derivative, and more particularly a C-glycoside such as C-β-D-xylopyranoside-n-propan-2-one, C-β-D-(3,4,5-triacetoxy)xylopyranoside-n-propan-2-one or C-β-D-xylopyranoside-2-hydroxypropan-2-one and derivatives thereof,
molecules or extracts that increase the intracellular level of ATP, for instance pyruvate and citrate,
molecules or extracts that ensure the protection and maintenance of the integrity of mitochondria, for instance an extract of Sene alata or of Laminaria digitata and peptides such as the hexapeptide Phe-Val-Ala-Pro-Phe-Pro,
molecules or extracts that stimulate the production of other chaperone proteins, for instance ectoin for HSP70, curcumin (diferuloylmethane) for HSP27, or an extract of Skeletonema costatum for HSP47.

Moreover, besides the combination of osmolytes according to the invention, the cosmetic composition comprises at least one cosmetically acceptable excipient, defined as a substance lacking a cosmetic effect per se, but which is useful for incorporating the active agent into the cosmetic composition or for formulating said composition.

This excipient may be chosen more particularly from pigments, colorants, polymers, surfactants, rheological agents, fragrances, electrolytes, pH modifiers, antioxidants and preserving agents, and mixtures thereof.

The cosmetic composition according to the invention may be, for example, a serum, a lotion, an emulsion, for example a cream, or alternatively a hydrogel, preferably a mask, or may be in the form of a stick or a patch, or alternatively a hygiene product for the scalp such as a shampoo or a hair conditioner, or alternatively a makeup product, in particular a composition intended to be applied to the nails, for example a nail varnish.

According to one variant of the invention, the abovementioned combination of osmolytes is encapsulated in lipid vesicles, for instance unilamellar or multilamellar liposomes.

The cosmetic composition comprising the lipid vesicles is itself preferably formulated in the form of an aqueous gel or of an emulsion of oil-in-water (0/W) type, in order to facilitate the diffusion of said active agents, i.e. the combination of osmolytes optionally combined with the abovementioned molecules or extracts, across the stratum corneum.

In the particular case in which the cosmetic composition comprising lipid vesicles is an oil-in-water emulsion, said vesicles are more particularly stabilized in the aqueous phase of the emulsion by adding to said aqueous phase a polysaccharide, more particularly by adding sodium alginate.

The cosmetic compositions according to the invention have a particularly desired effect for restoring, maintaining or reinforcing the skin moisturizing effect.

The cosmetic compositions according to the invention also have a particularly desired effect for protecting the skin against different types of stress.

Finally, the compositions have an effect on preventing or slowing down the appearance of the signs of aging of the skin.

A subject of the invention is also the use of a combination of at least two osmolytes chosen from the group comprising taurine or a derivative thereof, inositol, betaine and trehalose as an active agent in a cosmetic composition as defined previously, for restoring, maintaining or reinforcing the moisturization of the skin and/or for protecting the skin against different types of stress and/or for preventing or retarding the appearance of the signs of aging of the skin, or for attenuating the effects thereof, or alternatively for promoting cell or tissue longevity, said combination being used alone or combined with other cosmetically active agents as defined previously or as resulting from the description that follows, including the examples.

A subject of the invention is also a cosmetic care method comprising applying to at least one body zone in need thereof of an efficient amount of a composition comprising a cosmetic agent as previously defined or is defined later on. According to a particular feature said method is for moisturizing the skin, and/or for protecting it against any type of stress, and/or alternatively for producing an antiaging effect, which comprises the application to the part of the facial or bodily skin concerned of an effective amount of a combination of at least two osmolytes in a cosmetic composition as defined previously or as resulting from the description that follows, including the examples, which form an integral part of the invention.

Other aims, characteristics and advantages of the invention will emerge clearly in the light of the examples and especially of the examples of cosmetic compositions using said combination, which data are given simply as illustrations and shall not therefore in any way limit the scope of the invention.

The examples form an integral part of the invention for any characteristic that appears to be novel relative to any prior art.

In the examples, all the percentages are given on a weight basis, the temperature is in degrees Celsius, and the pressure is atmospheric pressure, unless otherwise indicated.

### EXAMPLES

### Example 1

### Demonstration of the Stimulation of Production of HSPA1A by Normal Human Epidermal Keratinocytes

Materials and Methods

Cell type: normal human keratinocytes (NHK) as an immersed monolayer, strongly post-confluent and at the third subculture.

Culture medium: low-calcium K-SFM (Invitrogen) supplemented with Epidermal Growth Factor (EGF Sigma) at 2.5 ng/ml, pituitary extract (EP Invitrogen) 2.5 μg/ml and gentamicin (Sigma) 25 μg/ml. For the treatments with the various compounds, this medium is replaced with K-SFM medium not supplemented with EGF and EP.

Treatment time: 24 hours with the various kosmotropes, and other molecules or extracts, and combinations thereof.

Test compounds: the taurine, D-trehalose, inositol and betaine (Sigma) are dissolved in the culture medium and tested at concentrations of 166 and 500 μg/ml, or, when this was not possible for reasons of solubility or cytotoxicity, at substantially lower concentrations.

The combination of these four compounds in proportions of 1/1/1/1 by weight is tested at the same concentrations.

Assays of the mRNA of HMSPA1A by RT-QPCR: at the end of the incubation period, the culture supernatants are removed and the cells, rinsed with PBS (Invitrogen), are covered with a solution of Tri-reagent (Sigma) and then frozen immediately at −80° C.

Extraction of the total RNA is performed according to the protocol of the Tri-reagent supplier (Sigma) and removal of the traces of DNA is performed with the DNA-free kit (Ambion). The reverse transcription reaction is performed using Oligo(dT) primers and the enzyme superscript II (Gibco).

The PCR (Polymerase Chain Reaction) reactions are performed using a Light Cycler (Roche Molecular Systems, Inc.) and according to the procedures recommended by the supplier. The pairs of primers used in this study allow amplification of the following fragments:

Homo sapiens heat shock protein HSP70 protein 1A (abbreviation HSPA1A; Gene bank NM 02045; fragment size 213 bp) and liver glyceraldehyde 3-phosphate dehydrogenase 1A (abbreviation G3PDH; Gene bank NM 02046; fragment size 269 bp) serving as the reference gene (caretaker gene) for determining the relative expression of the target HSPA1A.

The reaction medium (10 μl) is formed from the primers for the two target mRNAs (HSPA1A and G3PDH), 2.5 μl of tenfold-diluted cDNA, reaction mixture (Roche) containing the enzyme Taq DNA polymerase, SYBR Green I fluorochrome (which becomes incorporated into the double-stranded DNA during the elongation step), and MgCl₂.

The incorporation of fluorescence into the amplified DNA is measured continuously in the course of the PCR cycles. For the same used marker, the later a sample leaves (high number of cycles), the lower the initial number of copies of mRNA.

Normal human keratinocyte cultures are treated for 24 hours with noncytotoxic doses (166 and 500 μg/ml) of each of the four osmolytes separately: taurine, D-trehalose, inositol and betaine. The transcriptional expression of HMSPA1A is measured by RT-PCR.

The control (untreated cells) is normalized to correspond to 100% expression.

Results

Strong heterogeneity of response is demonstrated for these osmolytes. A moderate but significant stimulation of expression of HSPA1A of 171% and 139%, respectively, is observed with taurine, and of 142% and 181%, respectively, with D-trehalose.

On the other hand, no significant stimulation can be demonstrated with inositol (90% and 101%, respectively) or betaine (52% and 85%, respectively).

Unexpectedly, treatment of the keratinocytes with a mixture corresponding to a 1/1 combination by weight of taurine and inositol is identified as an activator of HSPA1A expression: +476% at 166 μg/ml.

This is likewise the case for a combination of taurine, inositol and betaine in a 1/1/1 weight ratio, which stimulates the expression of HSPA1A by +455% at 166 μg/ml.

This is also similarly the case for the combination of four osmolytes formed from taurine, D-trehalose, inositol and betaine in a 1/1/1/1 weight ratio, which stimulates the expression of HSPA1A very significantly more than that of each of the kosmotropic compounds taken individually (stimulation of 404% and 400% for respective doses of 166 and 500 μg/ml of the mixture).

Hypotaurine also significantly stimulates the expression of HSPA1A (+379% at 166 μg/ml), more efficiently than taurine.

These results demonstrate a synergistic effect of these combinations for stimulating the expression of HSPA1A, and the value of using these combinations as active agent in cosmetic compositions for moisturizing the skin, promoting tissue longevity, preventing or treating cell aging and improving the resistance of skin cells to stress.

### Example 2

### Moisturizing Lotion

The cosmetic composition below is a moisturizing lotion (percentages expressed on a weight basis relative to the weight of the composition):

| | |
|---|---|
| Trehalose | 0.05 |
| Inositol | 0.05 |
| Betaine | 0.05 |
| Taurine | 0.03 |
| D-Xylose | 0.05 |
| Other excipients (including fragrances, preserving agents) | qs |
| Water | qs 100 |

This composition is a lotion applied daily to facial skin to obtain the desired moisturizing effect.

### Example 3

### Day Cream for Normal Skin

The percentages are indicated on a weight basis relative to the weight of the final composition.

| | |
|---|---|
| Trehalose | 0.1 |
| Inositol | 0.1 |
| Betaine | 0.1 |
| Taurine | 0.06 |
| D-Xylose | 0.001 |
| Emulsified excipients (including fragrances, preserving agents) | qs |
| Purified water | qs 100 |

This composition is an oil-in-water emulsion including the synergistic combination of four osmolytes mentioned above and D-xylose.

The cream is applied daily to facial skin to obtain the desired moisturizing effect.

### Example 4

### Day Cream for Dry Skin

The percentages are indicated on a weight basis relative to the weight of the final composition.

| | |
|---|---|
| Trehalose | 0.1 |
| Inositol | 0.1 |
| Betaine | 0.1 |
| Taurine | 0.06 |
| D-Xylose | 0.001 |
| Emulsified excipients (including fragrances, preserving agents) | qs |
| Purified water | qs 100 |

This composition is an oil-in-water emulsion whose formulation is suited to dry skin. It thus includes among the excipients shea butter and jojoba esters whose nutrient, emollient and substantive properties make them particularly suitable for use in compositions intended for dry skin.

This composition is applied daily to facial skin to obtain the desired moisturization.

What is claimed:

1. A cosmetic composition comprising at least one cosmetic active agent comprising a combination of at least two osmolytes selected from the group consisting of taurine or a taurine component, inositol, betaine and trehalose.

2. The cosmetic composition of claim 1, comprising, as taurine component, hypotaurine.

3. The cosmetic composition of claim 1, wherein the trehalose is D-trehalose.

4. The cosmetic composition of claim 1, comprising a combination of taurine or a taurine component and inositol.

5. The cosmetic composition of claim 1, comprising a combination of taurine or a taurine component, inositol and betaine.

6. The cosmetic composition of claim 1, comprising a combination of taurine or a taurine component, D-trehalose, inositol and betaine.

7. The cosmetic composition of claim 1, further comprising D-xylose.

8. The cosmetic composition of claim 1, wherein the combination of osmolytes is encapsulated in lipid vesicles.

9. The cosmetic composition of claim 8, wherein the lipid vesicles are unilamellar or multilamellar liposomes.

10. The cosmetic composition of claim 8, wherein the lipid vesicles comprise amphiphilic lipids containing phosphatidylcholine.

11. The cosmetic composition of 10, wherein said amphiphilic lipids comprise lecithin.

12. The cosmetic composition of claim 1, wherein the weight ratio of each osmolyte in the combination relative to the weight of the osmolyte of the combination that is present in smallest amount in said combination is ranging between 1 and 10.

13. The cosmetic composition of claim 12, wherein said weight ratio of each osmolyte relative to the weight of the osmolyte present in smallest amount is between 1 and 5.

14. The cosmetic composition of claim 1, wherein the weight ratio of each osmolyte of the combination comprising taurine or a taurine component, inositol, betaine and trehalose is between 1/1/1/1 and 1/2/2/2.

15. The cosmetic composition of claim 1, comprising from 0.001% to 10%, by weight of the cosmetic active agent, of a cosmetically acceptable excipient.

16. The cosmetic composition of claim 15, formulated in the form of a serum, a lotion, an emulsion, a cream, a hydrogel, a mask, a stick, a patch, a hygiene product for the scalp, a shampoo, a hair conditioner, a makeup product, a nail composition, or a nail varnish.

17. The composition as claimed in claim 15, further comprising at least one of:
one or more other plant extracts or molecules with moisturizing properties;
at least one molecular constituent of natural moisturizing factor (NMF);
a lipid constituent selected from a cholesterol and cholesteryl sulfate; and
a fatty acid.

18. The composition of claim 17, wherein:
said plant extract or molecule with moisturizing properties is selected from the group consisting of a glycol, glycerol, a natural polyol, a natural or synthetic ceramide, and hyaluronic acid or a fragment thereof of lower molecular weight;
said molecular constituent of natural moisturizing factor (NMF) is selected from the group consisting of lactate, citrate, urea, a pyrrolidonecarboxilic acid salt, Na⁺, K⁺, Ca²⁺, Mg²⁺, serine, citrulline, alanine and threonine; and
said fatty acid is selected from the group consisting of omega-3, omega-6 and omega-9.

19. The composition as claimed in claim 15, further comprising at least one of:
a molecule or extract that stimulates the biosynthesis of the cutaneous aquaporins;
a molecule or extract that activates the biosynthesis of profilaggrin and filaggrin;
a molecule or extract that activates the biosynthesis of involucrin and transglutaminase of type 1;
a molecule or extract that activates caspase 14;
a molecule or extract that stimulates epidermal kallikreins;
a molecule or extract that stimulates the synthesis of epidermal lipids;
an antioxidant that protects epidermal lipids;
a molecule or extract that reinforces the skin barrier by stimulating the terminal differentiation of keratinocytes or transglutaminase, or both;
a molecule or extract that stimulates epidermal renewal;
a molecule or extract that protects the germinative compartment and the stem cells of the epidermis;
a molecule or extract that stimulates the expression of the beta-1 integrins or that promotes the adhesion of the germinative keratinocytes to the dermo-epidermal junction;
a molecule or extract that promotes the formation of tight junctions;
a molecule or extract that stimulates the expression of the receptor CD44 and the binding of its natural ligand, hyaluronic acid;
a molecule or extract that stimulates the synthesis of glycosaminoglycans (GAG) in the epidermis
a molecule or extract that increases the intracellular level of ATP;
a molecule or extract that ensures the protection and maintenance of the integrity of mitochondria; and
a molecule or extract that stimulates the production of other chaperone proteins, for instance ectoin for HSP70, curcumin (diferuloylmethane) for HSP27, or an extract of Skeletonema costatum for HSP47.

20. The composition of claim 19, wherein:
said molecule or extract that stimulates the biosynthesis of the cutaneous aquaporins is selected from the group consisting of an extract of Ajuga turkestanica, an extract of Vanda coerulea, retinoic acid and retinol, a combination of an extract of Malva sylvestris and of an extract of Centella asiatica that is rich in heterosides;
said molecule or extract that activates the biosynthesis of profilaggrin and filaggrin is an extract of Voandzeia subterranea seeds;
said molecule or extract that activates the biosynthesis of involucrin and transglutaminase of type 1 is a polyphenol selected from the group consisting of epigallocatechin-3 gallate and an extract containing same;
said molecule or extract that activates caspase 14 is selected from the group consisting of green tea polyphenol, epigallocatechin-3 gallate and an extract containing same;
said molecule or extract that stimulates epidermal kallikrein is an extract of nopal;
said molecule or extract that stimulates the synthesis of epidermal lipid is an extract of Helianthus annuus L. or Luffa cylindrica seeds;
said antioxidant that protects epidermal lipids is tocopherol or a derivative thereof; tocopherol palmitate; or tocopherol acetate;
said molecule or extract that reinforces the skin barrier is beta-ecdysone; a calcium derivative; or calcium gluconate;
said molecule or extract that stimulates epidermal renewal is corn oil;
said molecule or extract that protects the germinative compartment and the stem cells of the epidermis is tocopheryl phosphate;
said molecule or extract that stimulates the expression of beta-1 integrins or that promotes the adhesion of the germinative keratinocytes to the dermo-epidermal junction is magnesium aspartate or manganese chloride;
said molecule or extract that promotes the formation of tight junctions is an extract of Castanea sativa;
said molecule or extract that stimulates the expression of the receptor CD44 and the binding of its natural ligand is calcium gluconate,
said molecule or extract that stimulates the synthesis of glycosaminoglycans (GAG) is selected from a xylose derivative, a C-glycoside, a C-β-D-xylopyranoside-n-propan-2-one, C-β-D-(3,4,5-triacetoxy)xylopyranoside-n-propan-2-one or C-β-D-xylopyranoside-2-hydroxypropan-2-one and derivatives thereof;
said molecule or extract that increases the intracellular level of ATP is pyruvate and citrate;
said molecule or extract that ensures the protection and maintenance of the integrity of mitochondria is an extract of Sene alata, an extract of Laminaria digitata; a peptide, or hexapeptide Phe-Val-Ala-Pro-Phe-Pro;
said molecule or extract that stimulates the production of other chaperone proteins is ectoin for HSP70, curcumin (diferuloylmethane) for HSP27; or an extract of Skeletonema costatum for HSP47.

21. The composition of claim 15, in the form of an aqueous gel or an emulsion of oil-in-water type, wherein the combination of osmolytes is encapsulated in lipid vesicles.

22. The composition of claim 21, wherein the lipid vesicles are stabilized in the aqueous phase of said composition by adding a polysaccharide to said aqueous phase.

23. The composition of claim 15, wherein said composition is formulated for restoring, maintaining or reinforcing the moisturization of the skin; for protecting against different types of stress; for preventing or retarding the appearance of the signs of aging of the skin, or for attenuating the effects thereof; for promoting cell or tissue longevity.

24. A method for performing cosmetic care comprising applying to at least one body zone a sufficient amount of a cosmetic composition according to claim 1.

25. The method of claim 24, wherein said cosmetic care is selected from the group consisting of restoring, maintaining or reinforcing the moisturization of the skin; for protecting against different types of stress; for preventing or retarding the appearance of the signs of aging of the skin, or for attenuating the effects thereof; and for promoting cell or tissue longevity.

26. The method of claim 24, wherein said cosmetic composition comprises from 0.001% to 10%, by weight of said cosmetic active agent, of a cosmetically acceptable excipient.
